(19) United States
(12) Patent Application Publication
(10) Pub. No.: US 2016/0353995 A1
(43) Pub. Date: Dec. 8, 2016

(54) System and Method for Monitoring Fatigue

(71) Applicant: Under Armour, Inc., Baltimore, MD (US)

(72) Inventors: Mark A. Oleson, Baltimore, MD (US); F. Grant Kovach, Baltimore, MD (US); Nathan Dau, Baltimore, MD (US); Angela Nelligan, Baltimore, MD (US)

(21) Appl. No.: 14/730,506

(22) Filed: Jun. 4, 2015

(51) Int. Cl. A61B 5/0205; A61B 5/00

(52) U.S. Cl. CPC A61B 5/02055; A61B 5/7278; A61B 5/024

(57) ABSTRACT

A device includes a cardiac detecting component, a parameter detecting component, an interface component and a fatigue determining component. The cardiac detecting component detects a cardiac parameter of the user at a first time. The parameter detecting component detects a second parameter at a second time. The interface component receives a fatigue input from the user. The fatigue determining component generates a first fatigue indicator of the user based on the detected cardiac parameter at the first time, the detected second parameter at the second time and the fatigue input.

### BACKGROUND

The present invention generally deals with systems and methods for monitoring physical activity and fitness.

What is needed is a system and method for monitoring fatigue during exercise to avoid over-exercise and tissue damage.

### BRIEF SUMMARY OF THE DRAWINGS

The accompanying drawings, which are incorporated in and form a part of the specification, illustrate an exemplary embodiment of the present invention and, together with the description, serve to explain the principles of the invention. In the drawings:

FIG. 1 illustrates a conventional system for monitoring exercise using heart rate;

FIG. 2 illustrates a system in accordance with aspects of the present invention;

FIG. 3 shows a system in accordance with aspects of the present invention in training mode;

FIG. 4 shows the fatigue determining component in more detail as applied to the training mode;

FIG. 5 illustrates a process which describes the training mode steps;

FIG. 6 shows a system in accordance with aspects of the present invention in normal mode;

FIG. 7 illustrates the fatigue determining component in more detail as applied to a normal mode;

FIG. 8 shows a process which describes the normal mode steps;

FIG. 9 shows an example visual display method in accordance with aspects of the present invention for conveying fatigue information to the user.

### SUMMARY

The present invention is drawn to a system and method for monitoring fatigue during exercise such that results reflect subjective fatigue levels for each user, and for which more accurate results are obtained through a plurality of parameters thus significantly enhancing the ability for the user to exercise at optimum levels and to avoid over-exercise and tissue damage.

Various embodiments described herein are drawn to a device that includes a cardiac detecting component, a parameter detecting component, an interface component and a fatigue determining component. The cardiac detecting component detects a cardiac parameter of the user. An additional parameter detecting component detects an additional parameter. The interface component receives a fatigue input from the user. The fatigue determining component generates a fatigue indicator of the user based on the detected cardiac parameter, the detected additional parameter and the fatigue input. The interface component conveys fatigue indications to the user.

A plurality of cardiac parameter detection events, additional parameter detection events, accompanying fatigue input events and calculated fatigue indications for a user are accumulated over a period of time into a library. The calculated fatigue indications are then conveyed to the user through the interface component. The user may then use the indications to modify activity.

### EXAMPLE EMBODIMENTS

The concept of physical activity trackers grew out of written logs that led to spreadsheet-style computer logs in which entries were made manually, such as that provided in the US by the President's Council on Physical Fitness and Sports as part of The President's Challenge Improvements in technology in the late 20th and early 21st century have made it possible to automate the monitoring and recording of fitness activities and to integrate them into more easily worn equipment. Early examples of this technology include wristwatch-sized bicycle computers that monitored speed, duration, distance, etc., available at least by the early 1990s. Wearable heart rate monitors for athletes were available in 1981. Wearable fitness tracking devices, including wireless heart rate monitoring that integrated with commercial-grade fitness equipment found in gyms, were available in consumer-grade electronics by at least the early 2000s. Wearable fitness tracking computers with tightly integrated fitness training and planning software were available as consumer products by at least 2006.

Muscle fatigue is the decreased ability to generate appropriate amounts of muscle force or power during on-going contractile activity. Muscle fatigue may vary according to the cause or underlying mechanisms, with variable rates of recovery. For example, fatigue brought on by a marathon is different from fatigue that prevents a 10th bench press repetition at a given resistance. Exercise-induced muscle fatigue can occur shortly after onset of exercise (acute muscle fatigue) or after a constant, high-intensity exercise had been carried out for a prolonged period of time (delayed exercise-induced fatigue), which is characterized by tiredness only after a longer duration of constant exercise. Acute muscle fatigue is usually maximal immediately post-exercise. Exercise-induced muscle fatigue can develop under a sub-maximal as well as maximal intensity exercise. Exercise-induced fatigue can occur in healthy or diseased subjects, and depends on age, gender, physical fitness, mode and duration of exercise, and the underlying disease.

Fatigue as a functional sign and muscle damage as a structural sign can be observed after prolonged exercise like marathon running or after strenuous exercise, especially with the involvement of eccentric contractions. For fatigue due to prolonged exercise, hypoxic conditions and the formation of free oxygen radicals seem to be of causal importance, resulting in an elevated lysosomal activity. Eccentric exercise of high intensity rather results in a mechanical stress to the fibers. Although these different mechanisms can be discerned experimentally, both result in similar impairments of muscle function. A good training status may attenuate the clinical signs of fatigue and muscle damage. The symptoms and events occurring during delayed onset of muscle soreness can be explained by a cascade of events following structural damage to muscle proteins.

So, decreasing the force production during exercise can be regarded as a safety mechanism. If fatigue does not occur or is delayed, structural damage to muscle cells and supportive tissues due to fatigue would not occur during the workout. Monitoring physical activity, e.g. a workout, might therefore help fatigue avoidance.

Since it has been shown that there is a link between heart rate and fatigue, conventional systems and methods for monitoring a workout usually employ heart rate measurements.

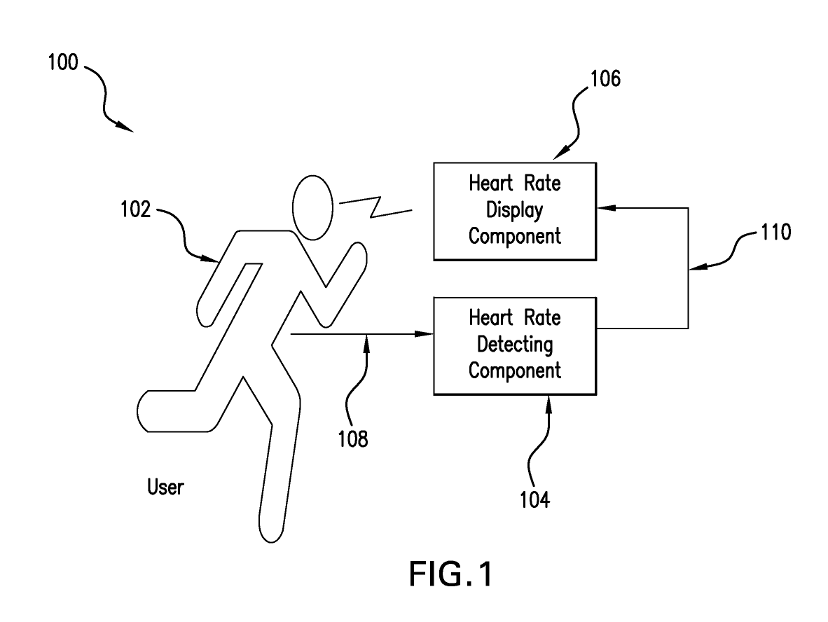

FIG. 1 illustrates a conventional system 100 for monitoring exercise by heart rate.

As shown in the figure, system 100 includes a user 102, a heart rate detecting component 104 and a heart rate display component 106.

Heart rate detecting component 104 is arranged to receive a signal from user 102 via line 108 and to output data via line 110 to heart rate display component 106.

Heart rate detecting component 104 is operable to convert a heart rate signal to data for display. Heart rate display component 106 is operable to visually display a heart rate number according to an input signal.

During activity of user 102, heart rate detecting component 104 detects the heart rate of the user as shown by arrow 108, and converts the detected heart rate signal to data. The data is received via line 110 by heart rate display component 106. Heart rate display component 106 then converts the data to a heart rate number for visual display user 102.

Since there is somewhat of a correlation between heart rate and fatigue, heart rate can and is used to monitor level of exercise activity and to signal possible adverse conditions so that over-exercise and resulting muscle damage can be avoided. However any specific heart rate result, when taken in isolation, can mean different exercise states for different persons and for different fitness and health levels. For example, for a specific heart rate, one person may feel fatigued while another while another healthier person may not be in a fatigue state at all. In addition, the fatigue heart rate for one of those persons may change as the person gets fitter. Furthermore, there are many other measurable parameters which are not used by conventional systems and methods which, when used in conjunction with heart rate, can achieve much better fatigue-indicating results.

One aspect in accordance with the present invention is drawn to the use of a component that detects an additional parameter in conjunction with a component that detects a cardiac parameter in order to monitor fatigue. As opposed to using a single parameter (as used conventionally and which may not be very accurate), a system and method in accordance with the present invention uses a cardiac parameter in addition to other parameters to determine the level of fatigue of a user. In particular, the fatigue level is determined based on the detected cardiac parameter and the additional parameters as compared to a priori thresholds indicating an unacceptable level of fatigue.

Another aspect of the invention is drawn to developing personalized fatigue indications. For example, in some embodiments, a pre-determined threshold level may be used when determining the level of fatigue of a user. A system may be trained by detecting cardiac and other parameters in addition to user fatigue inputs to establish a range of fatigue thresholds including, for instance, a threshold for an unacceptable level of fatigue. These fatigue thresholds may change based on a “training or learning” of the system through additional use by the user. These established user-unique fatigue thresholds are then used to determine future measurements of fatigue to be conveyed back to the user.

Another aspect in accordance with the present invention is drawn to the use of an interface component to allow the user to input that he is fatigued and to additionally input the level of fatigue he is feeling. This information is used along with the cardiac parameter and additional parameters which accompany the fatigue inputs to train the system to establish the personalized fatigue thresholds.

Another aspect in accordance with the present invention is drawn to the use of a fatigue determining component to process the cardiac parameters and additional parameters which accompany the user's fatigue inputs into the user-unique thresholds. Over time, during training of the system, a plurality of user inputs can be compiled into a stored “library” of calculated thresholds personalized to the user and which can be updated by the user in additional training periods to reflect changes in fitness and to increase accuracy. In some embodiments in accordance with the present invention, the library can store personalized fatigue information for multiple users.

Another aspect of the present invention is drawn to the use of the interface component to additionally provide a graphic user interface (GUI). After the system has been trained, the GUI easily informs a user of his level of fatigue so that he can use the information to adjust his level of activity in order to, for example, set a particular level of activity or to avoid over activity.

Conventional activity monitoring systems and methods measure and convey a single cardiac parameter such as heart rate to a user. Such a measurement conveys fatigue information only very loosely, since the heart rate when fatigued is different for different persons and for any one person can change with levels of health and fitness.

Systems and methods in accordance with the present invention overcome the limitations of conventional systems and methods by using user-subjective fatigue inputs along with user cardiac parameters in order to eliminate the differences in fatigue heart rate between users and between users' fitness levels. Furthermore, measurements for one or more additional parameters may be employed, which can greatly enhance accuracy as compared to a single parameter. In addition, accumulation of fatigue data in a “library” allows for a range of fatigue levels to be determined and allows even more accuracy to be achieved. Embodiments in accordance with the present invention also use methods of displaying fatigue levels to users which can help them anticipate and thus avoid the excessive levels, which may lead to the adverse consequences of over-exercise.

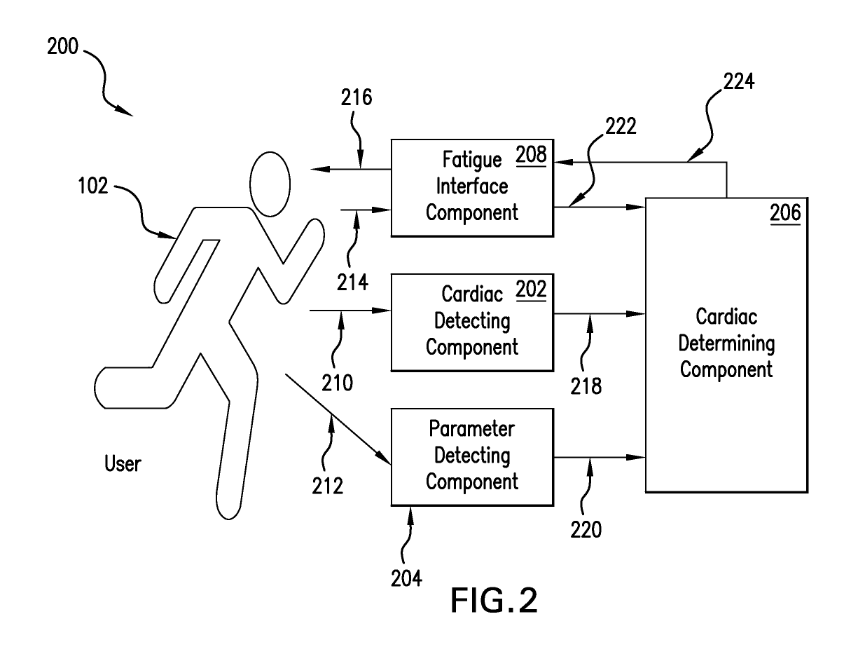

Aspects of the present invention will now be further described with reference to FIGS. 2-9.

FIG. 2 illustrates system 200, a system in accordance with aspects of the present invention.

As shown in the figure, system 200 includes a user 102, a cardiac detecting component 202, a parameter detecting component 204, a fatigue determining component 206 and a fatigue interface component 208.

Cardiac detecting component 202 is arranged to receive a cardiac signal from user 102 via a line 210 and to output cardiac data to fatigue determining component 206 via a line 218. Parameter detecting component 204 is arranged to receive a parameter signal from user 102 via a line 212 and to output parameter data to fatigue determining component 206 via a line 220. Fatigue interface component 208 is arranged to receive a fatigue input from user 102 via a line 214 and to output fatigue data to fatigue determining component 206 via a line 222. Fatigue determining component 206 is arranged to output fatigue indication data to fatigue interface component 208 via a line 224. Fatigue interface component is further arranged to output a fatigue indicator to user 102 via a user interface 216.

Cardiac detecting component 202 may be any system or device that is able to receive and detect cardiac parameter signals and to output a corresponding cardiac data. Non-limiting examples of a cardiac parameter include a heartbeat, a heartbeat amplitude, a heartbeat rhythm, a change in heartbeat amplitude, a change in heartbeat rhythm, a pulse, a change in pulse, an electrocardial signals, a change in an electrocardial signal and combinations thereof.

Parameter detecting component 204 may be any system or device that is able to receive and detect at least one other parameter signal and to output corresponding parameter data. Non-limiting examples of detected parameters include breathing rate, a change in gait, a biomarker and other parameters.

Fatigue interface component 208 may be any system or device that is able to receive a user fatigue and fatigue level commands and to output corresponding data. Fatigue and fatigue level commands may be input by any known system or method, non-limiting examples of which include pressing buttons on keyboard, a touch graphic user interface, a microphone or combinations thereof.

Fatigue determining component 206 may be any system or device that is able to receive cardiac and parameter data and to output fatigue indication data based on the cardiac and parameter data. Fatigue interface component 208 is further operable to convey a fatigue indication signal to a user via any known type of interface, non-limiting examples of which include a visual display, a sound, a contact vibration or combinations thereof.

In operation, presume for purposes of discussion that user 102 is exercising. A cardiac signal and a parameter signal are input via lines 210 and 212 respectively from user 102. Cardiac detecting component 202 receives the cardiac signal and parameter detecting component 204 receives the parameter signal, which are then passed as data to fatigue determining component 206 via lines 218 and 220, respectively. Fatigue interface component 208 accepts a subjective fatigue state and passes this to fatigue determining component 206 via line 222. Fatigue determining component 206 processes cardiac data, parameter data, fatigue state into thresholds that can be used to provide fatigue feedback to user 102. The feedback is sent as data via line 224 to fatigue interface component 208 which then conveys the fatigue information to user 102 via user interface 216.

It should be noted that the use of a parameter is not intended to be limiting and this parameter can include sound, a change in sound, proximity, change in proximity, location, a change in location position, velocity, acceleration, jerk, temperature, a change in temperature, moisture, a change in moisture, impedance, a change in impedance, resistance, a change in resistance, capacitance, a change in capacitance, inductance, a change in inductance, voltage, a change in voltage, current, a change in current, volume, a change in volume, pressure, a change in pressure, breathing rate, a change in breathing rate, breath, gait, a change in gait, cadence, a change in cadence, ground reaction time, a change in ground reaction time, ground contact time, a change in ground contact time, air time, a change in air time, hydration, a change in hydration, sound, a change in sound, a composition of matter, a change of composition of matter, biomarkers, changes in biomarkers, physiological parameters, changes in physiological parameters, an electromagnetic signal, a change in an electromagnetic signal, time, a change in time and combinations thereof.

Operation of system 200 occurs in two phases: a training phase and a normal phase.

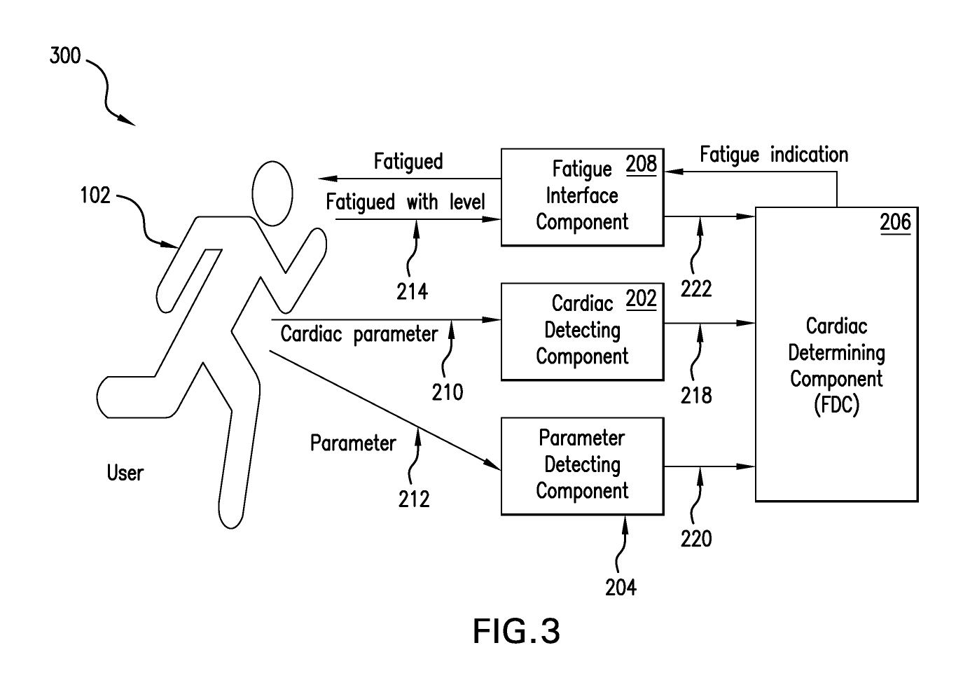

FIG. 3 illustrates system 300, which shows system 200 in operating in a training mode.

As shown in the figure, system 300 includes all the component of system 200, operable and arranged identically. In system 300, user 102 does not receive any fatigue feedback and so line 224 and line 216 are not used. Since these are the only differences, the components of system 300, their arrangements and operability will not be repeated here in the interests of brevity.

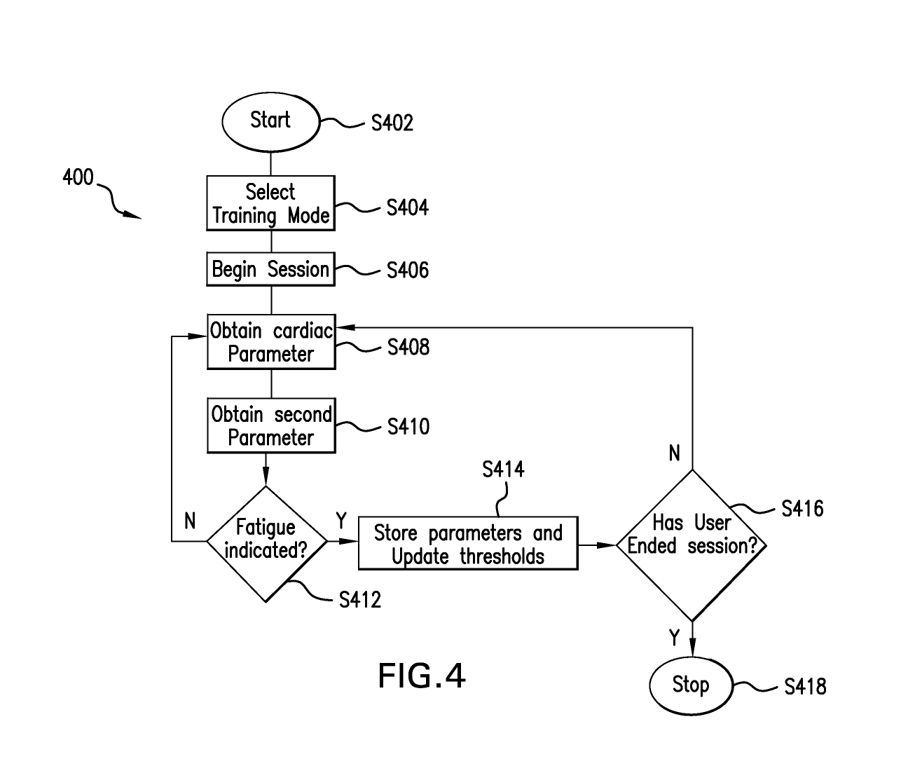

The operation of system 300 will now be further explained with additional reference to FIG. 4.

FIG. 4 shows a method 400 of operation for a training mode in accordance with aspects of the present invention.

As shown in the figure, method 400 starts at 5402, and user 202 selects a training mode (S404). For example, as shown in FIG. 2, user 102 may employ fatigue interface component 208. Fatigue interface component 208 would then inform fatigue determining component 206 that system 200 is to be in the training mode.

Returning to FIG. 4, user 202 then begins an exercise session (S406). For example, user may jog, lift weights, or perform some type of strenuous exertion.

While the user is exercising, a first cardiac parameter is detected (S408). For example, as shown in FIG. 3, cardiac detecting component 202 detects a cardiac signal via line 210. Cardiac detecting component 202 outputs cardiac data, corresponding to the detected cardiac signal, to fatigue determining component 206 via line 218.

Returning to FIG. 4, after the cardiac parameter is detected another parameter is detected (S410). For example, as shown in FIG. 3, parameter detecting component 204 detects a parameter signal via line 212. Parameter detecting component 204 outputs parameter data, corresponding to the detected parameter signal, to fatigue determining component 206 via line 220.

Returning to FIG. 4, it is then determined whether the user indicates fatigue (S412). For example, returning to FIG. 3, user 102 may provide input via fatigue interface component 208. In some embodiments, the input may have a binary form, e.g., fatigued/not fatigued. In some embodiments, the input may have a scaled form, e.g., a scale of 1-10, wherein an input of “1” may indicate that user 102 is not fatigued whereas an input of “10” may indicate that user 102 is the most fatigued. In some embodiments, the input may have a threshold form, e.g., a first threshold indicating that user 102 is experiencing sufficient fatigue and a second threshold indicating that user 102 is experiencing too much fatigue. It should be noted that any known system or method of indicating fatigue may be used in accordance with aspects of the present invention, and that these discussed embodiments are non-limiting example embodiments.

Returning to FIG. 4, if there is no fatigue input (N at S412), a new set of parameters are detected (return to S408). However, if there is fatigue input (Y at S412), then the data corresponding to the detected parameters is stored and thresholds are updated (S414). For example, as shown in FIG. 3, fatigue determining component 206 has received the cardiac and parameter data via lines 218 and 220, respectively, and has received the data representing the user input for fatigue via line 222.

At this point there are two detected parameters of user 102 in addition to a subjective input by user 102 regarding fatigue. For example, cardiac detecting component 202 may provide cardiac data corresponding to a detected heart rate of user 102, parameter detecting component 204 may provide parameter data corresponding to a respiration rate of user 102 and fatigue interface component 208 may input fatigue data corresponding to a fatigue indicator of “4,” on a scale of 1-10, as a subjective user assessment of the level of fatigue of user 102. Fatigue determining component 206 then generates the fatigue information based on a function of the cardiac data, the parameter data and the fatigue data. Fatigue determining component 206 additionally stores the fatigue information, and its association with the cardiac data, the parameter data and the fatigue data. In this manner, as will be described later, if similar cardiac data and parameter data are provided to fatigue determining component 206, a level of fatigue may be indicated to user 102 without user input.

Returning to FIG. 4, it is then determined whether the user has ended the exercise session (S416). Any known method of determining an end of an exercise session may be used. Some non-limiting examples of which include user 102 indicating an end of the exercise session via a GUI and a drastic and/or sudden decrease in a detectable parameter associated with the exercise session, e.g., acceleration, velocity, etc.

If the exercise session has not ended (N at S416) then a new set of parameters are detected (return to S408). If the exercise session has ended (Y at S416), then method 400 stops (S418).

In some embodiments, method 400 is implemented once to generate fatigue information for user 102. In some embodiments, method 400 may be implemented a plurality of times to generate a library of fatigue information for multiple instances of exercise related fatigue for user 102. In some embodiments, method 400 may be implemented a plurality of times for a plurality of users to generate a library of fatigue information for multiple instance of exercise related fatigue for the multiple users, wherein each user may be associated with respective fatigue information. In some embodiments, method 400 may be implemented a plurality of times for a plurality of users to generate a library of average fatigue information for multiple instance of exercise related fatigue for use by any user.

Method 400 may be repeated for user 102 to update fatigue data. In particular, as user 102 continues to exercise, user 102 will become less fatigued for a given amount of exercise. This change in fatigue as a function of exercise may be reflected in changes in the updated fatigue data.

At this point, user 102 can use system 200 in a normal mode of operation.

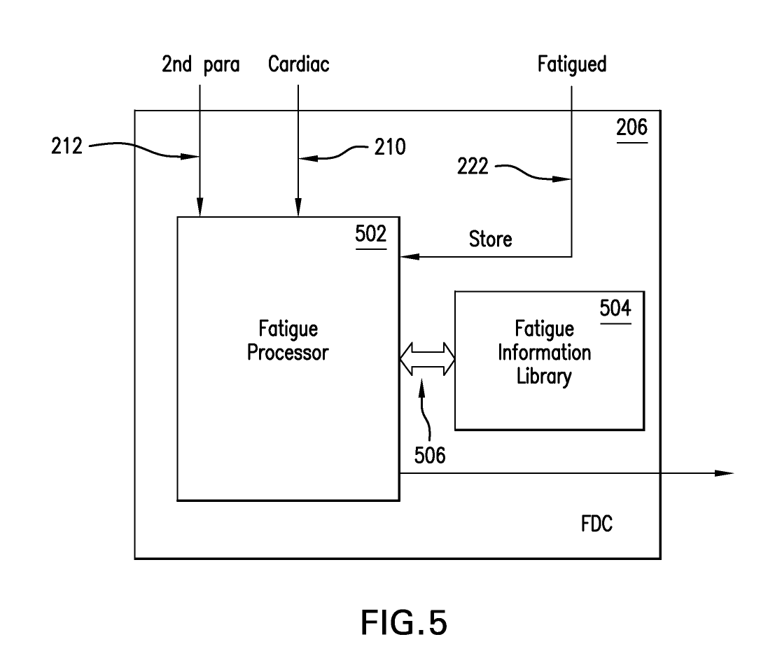

The above description has touched on the processing and storage of parameter and fatigue data into a library of fatigue information by fatigue determining component 206. A more detailed look at fatigue determining component 206 will now be provided with additional reference to FIG. 5.

FIG. 5 illustrates an example embodiment of fatigue determining component 206 in as applied to the training mode.

As shown in the figure, fatigue determining component 206 includes a fatigue processor 502 and a fatigue information library component 504.

Fatigue processor 502 is arranged to receive and transmit all the data and user inputs as received by fatigue determining component 206 and to transmit the output. Fatigue information library component 504 is arranged to connect to fatigue processor 502 via a bidirectional interface 506.

Fatigue processor 502 processes data and control of other components. Fatigue information library component 504 provides data storage.

In training mode, every time a user a fatigue input, the accompanying cardiac data, parameter data and fatigue data appearing via line 210, line 212 and line 222, respectively is written by fatigue processor 502 to fatigue information library component 504 for storage. Thus, over time, a library of fatigue information is compiled.

In this embodiment, raw data is stored and is later used by fatigue processor 502 to compute fatigue indications representing different levels of fatigue. This is not meant to be limiting. In other embodiments in accordance with aspects of the present invention, any useful data may be stored in fatigue library information component 504 and any fatigue indicator which is of use to the user may be computed by fatigue processor 504.

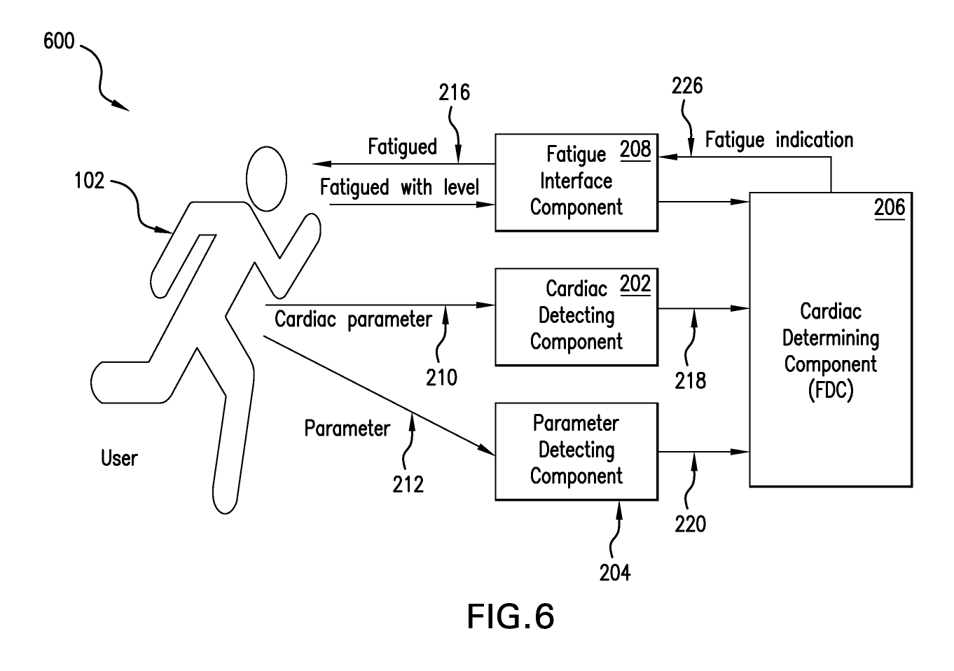

At this point, system 200 has a baseline for determining fatigue of user 102. In the embodiment discussed above with reference to FIG. 4, the baseline of fatigue is determined through use of system 200, wherein cardiac and other parameters are detected and the user provided fatigue input. However, in other embodiments, a baseline of fatigue may be initially provided in fatigue determining component 206. For example, a factory setting of fatigue may be provided in fatigue determining component 206, wherein the factory setting is based on cardiac and other detected parameters as associated with a group of users. For example, fatigue levels may be previously determined for the average male, female, person of a particular age, person of a particular age, etc. In either case, whether determined through use as discussed with reference to FIG. 4 or whether provided via a factory setting, the fatigue data within fatigue determining component 206 is a priori fatigue data that is used to determine fatigue of user 102 during subsequent exercise sessions. This will be further described with reference to FIG. 6. The normal mode of operation will now be explained.

FIG. 6 illustrates system 200 as operating in a normal mode.

As shown in the figure, user 102 does not give fatigue states but instead receives fatigue indications from the system and so line 224 and line 216 are now used but lines 214 and 222 are not. Since these are the only differences, the component listing of system 200, their arrangements and operability will not be repeated here in the interest of brevity.

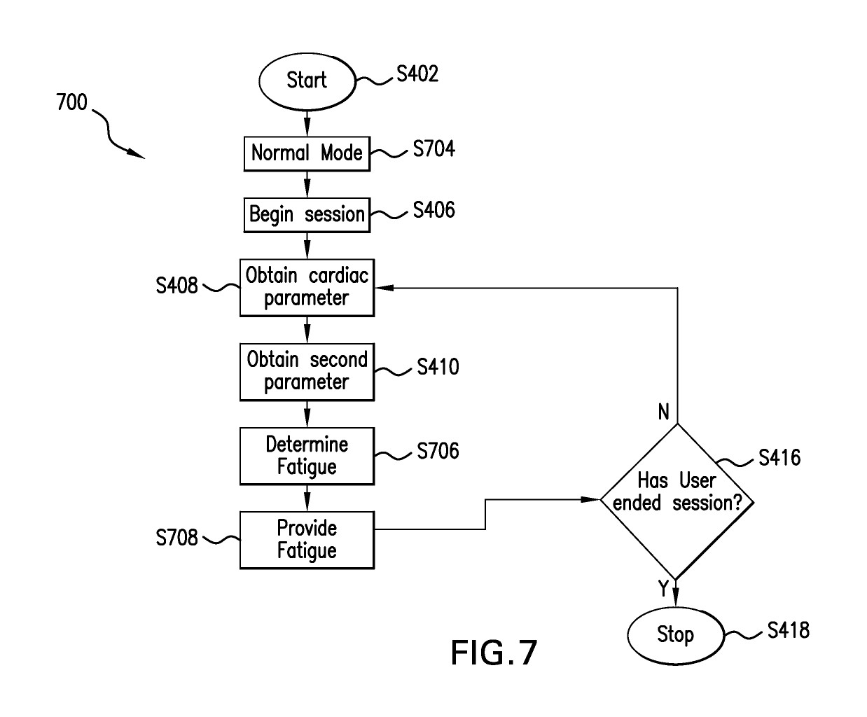

Operation of system 200 in a normal mode will now be explained with reference to FIG. 7.

FIG. 7 illustrates a method 700 of operation for normal mode in accordance with aspects of the present invention.

Method 700 begins (S402) and system 200 is in normal mode (S704). For example, as shown in FIG. 6, in some embodiments, system 200 may be in normal mode as a default mode. In some embodiments, user 102 may place system 200 in normal mode via a user interface on fatigue interface component 208.

Returning to FIG. 7, once in the normal mode, the user begins the exercise session (S406), system 200 obtains cardiac data (S408) and obtains parameter data (S410) all as discussed above with reference to method 400.

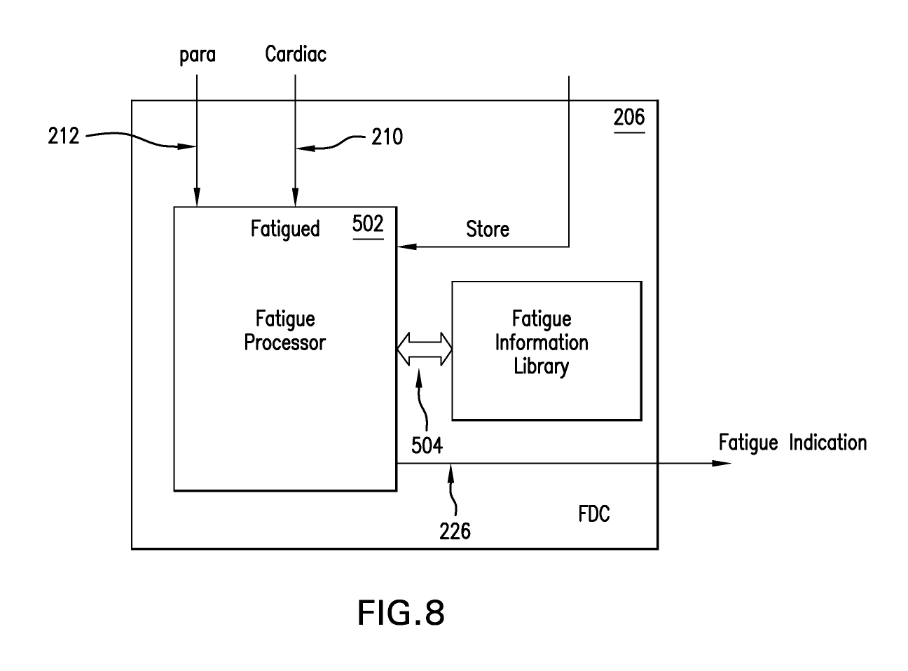

The fatigue of the user is then determined (S706). For example, returning to FIG. 6, fatigue determining component 206 uses the cardiac data and the parameter data to determine a corresponding user-tailored fatigue by using the a priori fatigue data. This will be described in greater detail with reference to FIG. 8.

FIG. 8 illustrates fatigue determining component 206 as applied to the normal mode.

As shown in the figure, for normal operation, fatigue processor 502 is receiving only cardiac and parameter data. These data are used by fatigue processor 502 to determine fatigue indications for display to user 102 and these indications are output via line 224.

Returning to FIG. 7, the fatigue data is then provided to the user (S708). For example, returning to FIG. 6, fatigue determining component 206 sends the determined fatigue data via line 224 to fatigue interface component 208 and interface component 208 passes it on via line 216. As already mentioned for FIG. 2, interface 216 may be a visual display, a sound, a contact vibration, or another manner or mechanism. In this example embodiment, fatigue interface component 216 provides this to the user using a visual indication.

Returning to FIG. 7, it is then determined whether the user has ended the session (S416). If not (N at S416), additional cardiac data is obtained (S408). If the session has ended (Y at S416), method 700 stops (S418).

While, for ease of explanation, the training and the normal modes have been described as separate phases of operation, other embodiments in accordance with the present invention may allow a single mode of operation where training inputs can be given in normal operation as fatigue indications are also displayed. In such embodiments, the accuracy of fatigue indications can be improved over time and there is no need to

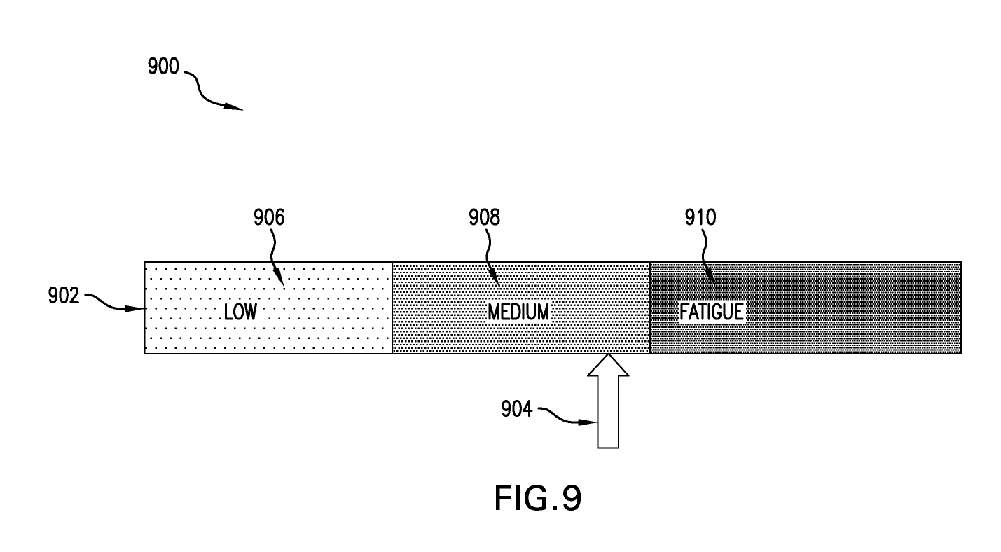

Conveying the determined fatigue indication to the user can be done by many manner or mechanism and it is not the intention for these to be limited. As mentioned earlier example embodiments in accordance with the present invention can include a visual interface, sound, vibration, or any other suitable method. For completeness, one of these example embodiments will be described below with reference to FIG. 9.

FIG. 9 shows display 900, a visual display method for conveying fatigue information to the user.

As shown in the figure, display 900 includes display graphic 902 and a pointer 904. Display graphic 902 includes a low region 906, a medium region 908 and a fatigue region 906.

In operation, pointer 904 is indicating to the user a visual representation of the fatigue level determined by fatigue determining component 206 of FIG. 2. It does this against display graphic 902 which represents a range of possible fatigue levels. As each cardiac and second parameter measurement is detected, the fatigue level and pointer position is updated.

Users can use this display to personalize their exercise regimens according to their own fatigue behaviors. The user might use this display to optimize exercise and avoid over-exercise and muscle damage by keeping the pointer, for instance, just below the fatigue region or by minimizing the time into that region. At any time, users can update their personal fatigue profile by re-entering the training mode.

In this example embodiment, a grey scale and outlined regions are used with a pointer to indicate fatigue level but this is not meant to be limiting for visual display methods. Other embodiments in accordance with the present invention could use other means of visual display such as colors, numbers, symbols and other suitable means. In addition, for emphasis, redundancy or other reasons, a visual display could be used in conjunction with one or more other methods of conveying fatigue information such as sound or vibration.

In summary, conventional activity monitoring devices provide only very limited information relating to fatigue level during exercise. Conventionally, they use only heart rate for display to the user, a parameter which is only very loosely correlated to fatigue level. Conventional systems do not take account of differences between persons, or, for any one person, for differences in fitness or health over time.

Embodiments in accordance with aspects of the present invention overcome all the stated limitations of a conventional heart rate monitor. By incorporating the detection of a plurality of parameters, said embodiments can take advantage of the wealth of additional information available from other measurements to significantly enhance the accuracy and usefulness of a fatigue level determination. By incorporating a training mode using user-generated subjective fatigue input, fatigue determination can be personalized to individuals and to their changes in fitness. Through accumulation of data into a library, accuracy is further enhanced. By conveying current fatigue level on a visual scale, over-exercise can be held back or avoided and the adverse effects of fatigue can be better controlled or eliminated.

In the drawings and specification, there have been disclosed embodiments of the invention and, although specific terms are employed, they are used in a generic and descriptive sense only and not for purposes of limitation, the scope of the invention being set forth in the following claims.

What is claimed as new and desired to be protected by Letters Patent of the United States is:

1. A device for use by a user, said device comprising:
a cardiac detecting component operable to detect a cardiac parameter of the user at a first time and to generate cardiac data based on the detected cardiac parameter;
a parameter detecting component operable to detect a second parameter at a second time and to generate fatigue data based on the detected second parameter;
an interface component operable to receive a fatigue input from the user; and
a fatigue determining component operable to generate a first fatigue indicator of the user based on the cardiac data, the fatigue data and the fatigue input.

2. The device of claim 1,
wherein said cardiac detecting component is further operable to detect the cardiac parameter at a third time,
wherein said parameter detecting component is further operable to detect the second parameter at a fourth time, and
wherein said fatigue determining component is operable to generate the first fatigue indicator of the user additionally based the detected cardiac parameter at the third time, the detected second parameter at the fourth time and the fatigue input.

3. The device of claim 2,
wherein said cardiac detecting component is further operable to detect the cardiac parameter of the user at a fifth time,
wherein said parameter detecting component is further operable to detect the second parameter at a sixth time, and
wherein said fatigue determining component is operable to generate a second fatigue indicator of the user based on the detected cardiac parameter at the fifth time, the detected second parameter at the sixth time and the first fatigue indicator.

4. The device of claim 3,
wherein the first time comprises the second time,
wherein the third time comprises the fourth time, and
wherein the fifth time comprises the sixth time.

5. The device of claim 1, wherein said second parameter detecting component is operable to detect, as the second parameter, one of the group selected from sound, a change in sound, proximity, change in proximity, location, a change in location position, velocity, acceleration, jerk, temperature, a change in temperature, moisture, a change in moisture, impedance, a change in impedance, resistance, a change in resistance, capacitance, a change in capacitance, inductance, a change in inductance, voltage, a change in voltage, current, a change in current, volume, a change in volume, pressure, a change in pressure, breathing rate, a change in breathing rate, breath, gait, a change in gait, cadence, a change in cadence, ground reaction time, a change in ground reaction time, ground contact time, a change in ground contact time, air time, a change in air time, hydration, a change in hydration, sound, a change in sound, a composition of matter, a change of composition of matter, biomarkers, changes in biomarkers, physiological parameters, changes in physiological parameters, an electromagnetic signal, a change in an electromagnetic signal, time, a change in time and combinations thereof.

6. The device of claim 1, wherein said parameter detecting component is operable to detect the second parameter over a period of time as the second time.

7. The device of claim 1, wherein said interface portion is further operable to provide a prompt to the user to quantify fatigue of the user.

8. A method comprising:
detecting, via a cardiac detecting component, a cardiac parameter of a user at a first time;
generating, via the cardiac detecting component, cardiac data based on the detected cardiac parameter;
detecting, via a parameter detecting component, a second parameter at a second time;
generating, via the parameter detecting component, fatigue data based on the detected second parameter;
receiving, via an interface portion, a fatigue input from the user; and
generating, via a fatigue determining component a first fatigue indicator of the user based on based on the cardiac data, the fatigue data and the fatigue input.

9. The method of claim 8, further comprising:
detecting, via the cardiac detecting component, the cardiac parameter at a third time;
detecting, via the parameter detecting component, the second parameter at a fourth time; and
generating, via the fatigue determining component, the first fatigue indicator of the user additionally based the detected cardiac parameter at the third time, the detected parameter at the fourth time and the fatigue input.

10. The method of claim 9, further comprising:
detecting, via the cardiac detecting component, the cardiac parameter of the user at a fifth time;
detecting, via the parameter detecting component, the second parameter at a sixth time; and
generating, via the fatigue determining component, a second fatigue indicator of the user based on the detected cardiac parameter at the fifth time, the detected parameter at the sixth time and the first fatigue indicator.

11. The method of claim 10,
wherein the first time comprises the second time,
wherein the third time comprises the fourth time, and
wherein the fifth time comprises the sixth time.

12. The method of claim 8, wherein said detecting, via a parameter detecting component, a parameter at a second time comprises detecting, as the second parameter, one of the group selected from sound, a change in sound, proximity, change in proximity, location, a change in location position, velocity, acceleration, jerk, temperature, a change in temperature, moisture, a change in moisture, impedance, a change in impedance, resistance, a change in resistance, capacitance, a change in capacitance, inductance, a change in inductance, voltage, a change in voltage, current, a change in current, volume, a change in volume, pressure, a change in pressure, breathing rate, a change in breathing rate, breath, gait, a change in gait, cadence, a change in cadence, ground reaction time, a change in ground reaction time, ground contact time, a change in ground contact time, air time, a change in air time, hydration, a change in hydration, sound, a change in sound, a composition of matter, a change of composition of matter, biomarkers, changes in biomarkers, physiological parameters, changes in physiological parameters, an electromagnetic signal, a change in an electromagnetic signal, time, a change in time and combinations thereof.

13. The method of claim 8, wherein said detecting, via a parameter detecting component, a parameter at a second time comprises detecting the second parameter over a period of time as the second time.

14. The method of claim 8, further comprising providing, via interface portion, a prompt to the user to quantify fatigue of the user.

15. A non-transitory, tangible, computer-readable media having computer-readable instructions stored thereon, the computer-readable instructions being capable of being read by a computer and being capable of instructing the computer to perform the method comprising:
detecting, via a cardiac detecting component, a cardiac parameter of a user at a first time;
generating, via the cardiac detecting component, cardiac data based on the detected cardiac parameter;
detecting, via a parameter detecting component, a second parameter at a second time;
generating, via the parameter detecting component, fatigue data based on the detected second parameter;
receiving, via an interface portion, a fatigue input from the user; and
generating, via a fatigue determining component a first fatigue indicator of the user based on based on the cardiac data, the fatigue data and the fatigue input.

16. The non-transitory, tangible, computer-readable media of claim 15, the computer-readable instructions being capable of being read by a computer and being capable of instructing the computer to perform the method further comprising:
detecting, via the cardiac detecting component, the cardiac parameter at a third time;
detecting, via the parameter detecting component, the second parameter at a fourth time; and
generating, via the fatigue determining component, the first fatigue indicator of the user additionally based the detected cardiac parameter at the third time, the detected parameter at the fourth time and the fatigue input.

17. The non-transitory, tangible, computer-readable media of claim 16, the computer-readable instructions being capable of being read by a computer and being capable of instructing the computer to perform the method further comprising:
detecting, via the cardiac detecting component, the cardiac parameter of the user at a fifth time;
detecting, via the parameter detecting component, the second parameter at a sixth time; and
generating, via the fatigue determining component, a second fatigue indicator of the user based on the detected cardiac parameter at the fifth time, the detected parameter at the sixth time and the first fatigue indicator.

18. The non-transitory, tangible, computer-readable media of claim 17,
wherein the first time comprises the second time,
wherein the third time comprises the fourth time, and
wherein the fifth time comprises the sixth time.

19. The non-transitory, tangible, computer-readable media of claim 15, wherein the computer-readable instructions are capable of instructing the computer to perform the method such that said detecting, via a parameter detecting component, a parameter at a second time comprises detecting, as the second parameter, one of the group selected from sound, a change in sound, proximity, change in proximity, location, a change in location position, velocity, acceleration, jerk, temperature, a change in temperature, moisture, a change in moisture, impedance, a change in impedance, resistance, a change in resistance, capacitance, a change in capacitance, inductance, a change in inductance, voltage, a change in voltage, current, a change in current, volume, a change in volume, pressure, a change in pressure, breathing rate, a change in breathing rate, breath, gait, a change in gait, cadence, a change in cadence, ground reaction time, a change in ground reaction time, ground contact time, a change in ground contact time, air time, a change in air time, hydration, a change in hydration, sound, a change in sound, a composition of matter, a change of composition of matter, biomarkers, changes in biomarkers, physiological parameters, changes in physiological parameters, an electromagnetic signal, a change in an electromagnetic signal, time, a change in time and combinations thereof.

20. The non-transitory, tangible, computer-readable media of claim 20, wherein the computer-readable instructions are capable of instructing the computer to perform the method such that said detecting, via a parameter detecting component, a parameter at a second time comprises detecting the second parameter over a period of time as the second time.
